(19) United States
(12) Patent Application Publication
(10) Pub. No.: US 2017/0042631 A1
(43) Pub. Date: Feb. 16, 2017

(54) INTRA-OPERATIVE MEDICAL IMAGE VIEWING SYSTEM AND METHOD

(71) Applicant: SURGERATI, LLC, Royal Oak, MI (US)

(72) Inventors: Florence Xini Doo, Royal Oak, MI (US); David C. Bloom, Chelsea, MI (US)

(21) Appl. No.: 15/306,214

(22) PCT Filed: Apr. 21, 2015
(86) PCT No.: PCT/US15/26916

§ 371 Date: Oct. 24, 2016

Related U.S. Application Data
(60) Provisional application No. 61/982,787, filed on Apr. 22, 2014.

(51) Int. Cl. A61B 90/00; H04N 13/04

(52) U.S. Cl. CPC A61B 90/37; H04N 13/044; H04N 13/0497; A61B 2017/00203

(57) ABSTRACT

An intra-operative medical image viewing system can allow a surgeon to maintain a viewing perspective on the patient while calling-up visual images on-the-fly. A digital image source has at least one image file representative of an anatomical or pathological feature of a patient. A display is worn by the surgeon or positioned between the surgeon and her patient during surgery. The display is selectively transparent, and exhibits to the surgeon an image derived from the image file. An image control unit retrieves the image file from the image source and controls the display so that at least a portion of the image depiction can be exhibited and modified at will by the surgeon. A plurality of peripheral devices are each configured to receive an image control input from the surgeon and, in response, generate an image control signal. Each peripheral accepts a different user-interface modality.

### CROSS REFERENCE TO RELATED APPLICATIONS

This application claims priority to Provisional Patent Application No. 61/982,787 filed Apr. 22, 2014, the entire disclosure of which is hereby incorporated by reference and relied upon.

### BACKGROUND OF THE INVENTION

Field of the Invention

The invention relates generally to generating, processing, transmitting or transiently displaying images in a medical environment, in which the local light variations composing the images may change with time, and more particularly to subject matter in which the image includes portions indicating the three-dimensional nature of the original object.

Description of Related Art

In a surgical environment, there are often many display screens each displaying different visual information that is of interest to the medical practitioner, such as a surgeon. In particular, the visual information may include images representing an anatomical or pathological feature of a patient, such as an X-ray, MRI, ultrasound, thermal image or the like. The term surgeon is used throughout this patent document in a broad sense to refer to any of the one or more specialized medical practitioners present in a surgical or interventional-procedural environment that provide critical personal treatment to a patient. In addition to practitioners and interventionalists, the term surgeon can also mean a medical student, as well as any other suitable person. The term surgical environment is also used broadly to refer to any surgical, interventional or procedural environment. Similarly, the term surgical procedure is chosen to broadly represent both interventional and non-interventional activities, i.e., including purely exploratory activities. FIG. 1 is a simplified illustration of a surgical environment in which numerous display screens 20, 22, 24 compete for the attention of a surgeon 26 while the surgeon provides critical personal treatment to a patient 28. The display screens 20, 22, 24 are typically located in widely distributed locations within the operating room. Some of the displays 22, 24 are suspended from boom-arms, others are mounted to the wall, and still others 20 can be mounted to mobile carts. An operating room that is filled with many display screens all presenting different relevant anatomical or pathological image data to the surgeon causes several problems in the medical community, which problems have proven particularly difficult to eradicate.

A first problem relates to distraction of the surgeon's attention posed by the need to frequently look away from her patient in order to see the images on one or more display screens dispersed about the operating room. While surgeons are generally gifted with extraordinary eye-hand coordination, the surgical procedures they perform often depend on sub-millimeter-level control of their instruments. The risk of a tiny, unwanted hand movement rises each time a surgeon must consult an image on a screen that is located some distance away from the patient. The accidental nicking of an adjacent organ could perhaps in some cases be attributed to the surgeon's momentary head turn as she looks at an important anatomical or pathological image on a display screen on a nearby medical cart or suspended from a boom arm.

A second problem that is provoked by the presence of multiple display screens in an operating room relates to compounding a surgeon's cognitive load. Cognitive load refers to the total amount of mental effort being used in the working memory of the surgeon. Surgeons are trained to function at high cognitive loading levels, yet every human has a limit. Biomedical research has confirmed that managing a surgeon's cognitive load level will allow her to perform at peak ability for a longer period of time. In operating room settings, one of the most intense contributors to the cognitive load of a surgeon is the mental act of image registration. Image registration is the process of transforming different sets of data into one coordinate system. For the surgeon in an operating environment, this means the ability to compare or integrate the data obtained from medical images presented on the display screens to the patient in front of them. For example, if the image on the display screen was taken (or is being rendered) from a perspective different than the instantaneous visual perspective of the surgeon, the surgeon automatically aligns the image to the patient by envisioning a rotation, pan, tilt, zoom or other manipulation of the displayed image to that of the live patient in front of them. While image registering a single static image to the patient may not be particularly taxing, the cognitive load quickly compounds when there are many display screens to be consulted, each exhibiting an image taken from yet a different perspective or presented in a different scale. Therefore, the multiplied act of image-registering a large number of images profoundly intensifies the cognitive loading imposed on a surgeon, which in turn produces an accelerated fatiguing effect.

Yet another problem that is provoked by the presence of multiple display screens in an operating room relates to ergonomics. Namely, the occupational safety and health of a surgeon is directly compromised by the required use of many widely-dispersed images during a surgical procedure. During a surgical procedure, which can sometimes last for many hours, the surgeon 26 must often look up from the patient 28 in order to obtain information from the various display screens 20, 22, 24. In the exemplary illustration of FIG. 1, if the surgeon 26 is required to gaze intently at the display screen 20 for a long period of time, her head must be held steadily in an uncomfortable sideways-looking direction. Some surgical procedures, such as a laparoscopic procedure for example, require the surgeon to watch the real-time image feed from a remote camera. The surgeon's gaze may be intently directed to the real-time image on a display screen for an extended period of time. Surgery does not afford the practitioner with the ability to rest or change positions at will in order to combat muscle cramps or nerve aggravations. On a daily basis, this physical fatigue limits a surgeon's ability to perform at optimum ability during long shifts. Over time, the stresses placed on the surgeon accumulate to the point where the injuries accumulate/compound and become chronic and must either be remediated through medical intervention or the surgeon prematurely limits (or truncates) her service career.

Furthermore, these problems can be inter-related. Issues associated with cognitive load and ergonomics compound each other to diminish a surgeon's working efficiency, which affect the patient by increasing the length of time they must undergo a surgical procedure. Naturally, increased procedure time impacts the surgeon's health but also the surgeon's productivity. That is, with more time in each surgery the surgeon can do fewer operations over the course of a year, which also then limits the surgeon's ability to gain experience. Increased procedure time impacts the patient in a number of ways also, including increased risks associated with prolonged time under anesthesia and its after-affects, increased risk for infections attributed to longer open incision times, longer hospital stays, increased medical costs, and the like.

Finding a solution to these persistent image-related problems in the operating room has been elusive. One reason is that any proposed solution must itself have a practical chance of being adopted in the surgical community. That is to say, a solution that works only in the lab or only for a small sub-set of practitioners will not be genuinely viable as a marketable product. A real solution needs to be practical for the medical community as a whole. Therefore, understanding and accommodating the medical community, as a whole, is a critical step in assessing whether or not a particular solution will have authentic merit. As a group, surgeons tend to be somewhat unique in temperament. They are generally recognized as excessively driven toward achievement, decisive, well organized, hardworking, assertive, and aim to reduce uncertainty in their operations to reduce risk for their patient's outcomes. Any touted ergonomical or cognitive load benefit (and resultant benefit to patient outcomes) weighs against the heavy judgment of centuries of historic medical science and knowledge. Medical students, and the physicians they become, learn from their mentors the tried and true methods and techniques of their predecessors to ensure no patient harm. Thus, the point of mentioning this assessment is that surgeons by and large will tend not to accept into their practice a new technique or new technology unless that new technology is regarded as practical. But not all surgeons are alike, and what may be regarded by one surgeon as practical will be deemed unacceptably impractical by another. Therefore, any attempt to introduce a solution to the above-mentioned image issues must be instantly perceived as being practicable to all (or at least a substantial majority of) surgeons. It is predictable that a majority of surgeons will not adopt a solution if the solution is perceived to be overly complicated or as requiring a high degree of training to master.

The reason why multiple display screens litter the typical operation room today is that display screens are universally intuitive. The mere act of looking at an image displayed on a screen requires no training for use. Therefore, if the surgeon needs to see more patient images during a surgical procedure, there is a tendency to add another display screen in the operating room. Adding more display screens, in turn, compounds the distraction, cognitive loading and ergonomic issues. A degenerative spiral results, because the current state of the art has no simpler, more intuitive option than adding more display screens to exhibit patient medical images in an operating room.

There is therefore a need for an improved system in which the customary multitude of medical images needed to be viewed by a surgeon during an operation are better managed so that a surgeon is not required to look away from the patient, so that the surgeon does not have to sustain heavy cognitive loading in order to mentally register all of exhibited images, and so that the surgeon does not suffer unnecessary additional physical stresses. However, any an improved system to overcome these issues must be easily and intuitively implemented without the need for extensive training or practice.

### BRIEF SUMMARY OF THE INVENTION

In summary, the invention is an intra-operative medical image viewing system that can allow the surgeon to maintain a viewing perspective on the patient while concurrently obtaining relevant information about the patient. The intra-operative medical image viewing system can include an image source having at least one image file representative of an anatomical or pathological feature of a patient. The intra-operative medical image viewing system can also include a display positionable between a surgeon and the patient during surgery. The display can be configured to exhibit and position at least one image to the surgeon overlaid on or above the patient. The intra-operative medical image viewing system can also include an image control unit configured to retrieve the image file from the image source and control the display so as to exhibit and modify at least a portion of the image. The intra-operative medical image viewing system can also include a plurality of peripheral devices. Each peripheral device may be configured to receive an image control input from the surgeon and, in response, generate an image control signal in a respective user-interface modality. The image control input can be representative of a desire by the surgeon to modify the at least one image exhibited by the display. Each peripheral device can define a different user interface modality.

In another aspect of the invention, an intra-operative medical image viewing system can include an image source having at least one image file representative of an anatomical or pathological feature of a patient or of a surgical implementation, trajectory or plan. The intra-operative medical image viewing system can also include a display positionable between a surgeon and the patient during surgery. The display can be configured to exhibit the image to the surgeon overlaid on the patient. The intra-operative medical image viewing system can also include an image control unit configured to retrieve the image file from the image source and control the display to exhibit and modify at least a portion of the image. The intra-operative medical image viewing system can also include at least one peripheral device configured to receive an image control input from the surgeon and in response transmit an image control signal to the image control unit. The image control input can be representative of a desire by the surgeon to modify the image exhibited by the display. The image control unit can be configured to modify the image in response to the image control signal in any one of a plurality of different three-dimensional modalities.

In another aspect of the invention, an intra-operative medical image viewing system can include an image source having an image file representative of an anatomical feature of a patient. The intra-operative medical image viewing system can also include a display wearable by a surgeon during surgery on the patient. The display can be selectively transparent and configured to exhibit an image to the surgeon overlaid on the patient. The intra-operative medical image viewing system can also include an image control unit configured to retrieve the image file from the image source and control the display to exhibit and modify the image. The image can be a visual representation of the anatomical feature of the patient. The image control unit can be responsive to inputs from the surgeon to modify the image to allow the surgeon to selectively position, size and orient the image exhibited on the display to a selectable first configuration. The intra-operative medical image viewing system can also include a station-keeping module. The station-keeping module can include a position module configured to detect a first position of the display when the first configuration can be selected and determine a change in position of the display from the first position. The station-keeping module can also include an orientation module configured to detect a first orientation of the display when the first configuration can be selected and determine a change in orientation of the display from the first orientation. The station-keeping module can also include a registration module configured to determine a registration default condition that can be defined by a frame of reference or a coordinate system; the first configuration, the first position, and the first orientation can also be defined the frame of reference or the coordinate system. The station-keeping module can also include an image recalibration module configured to determine one or more image modification commands to be applied by the display to change the image from the first configuration to a second configuration in response to at least one of the change in position and change in the orientation. The image recalibration module can be configured to transmit the one or more image modification commands to the image control unit and the image control unit to control the display in response to the one or more image modification commands and change the image to a second configuration. The second configuration can be different from the first configuration and consistent with the registration default condition.

The present invention is particularly adapted to manage the multitude of medical images needed to be viewed by a surgeon during an operation so that a surgeon is not required to look away from the patient, so that the surgeon does not have to sustain heavy cognitive loading in order to mentally register all of exhibited images, and so that the surgeon does not suffer unnecessary additional physical stresses. In addition, the present invention can be easily and intuitively implemented without the need for extensive training or practice. By lowering distraction, cognitive loading, and concomitant fatigue, use of the present invention will lead to greater efficiency. That is to say, the surgeon can perform more procedures per shift, so that her productivity is improved. In addition, a surgeon executing a surgical procedure with the present invention will be more productive, learn faster and perform better, thereby leading to greater effectiveness.

### BRIEF DESCRIPTION OF THE SEVERAL VIEWS OF THE DRAWINGS

These and other features and advantages of the present invention will become more readily appreciated when considered in connection with the following detailed description and appended drawings, wherein:

FIG. 1 is a perspective view of a surgical environment according to the prior art;

FIG. 2 is a perspective view of an embodiment of the invention in a first surgical environment;

FIG. 3 is a schematic view of an embodiment of the invention in a second surgical environment;

FIG. 4 is another schematic view of the invention;

FIG. 5 is a perspective view of an embodiment of the invention in a third surgical environment;

FIG. 6 is a perspective view of an embodiment of the invention in a fourth surgical environment;

FIG. 7 is a perspective view of an embodiment of an embodiment of the invention in a fifth surgical environment;

FIG. 8 is a perspective view of a two-dimensional image in a planar configuration;

FIG. 9 is a perspective view of the two-dimensional image of FIG. 8 in a wrapped configuration;

FIG. 10 is a perspective view of a two-dimensional image in a planar configuration in two different observable planes;

FIG. 11 is a series of three-dimensional tomographic slices of an anatomical feature of a patient;

FIG. 12 is a perspective view of an embodiment of the invention in a sixth surgical environment;

FIG. 13 is a perspective view of an embodiment of the invention in a seventh surgical environment; and

FIG. 14 is a perspective view of an embodiment of the invention in an eighth surgical environment.

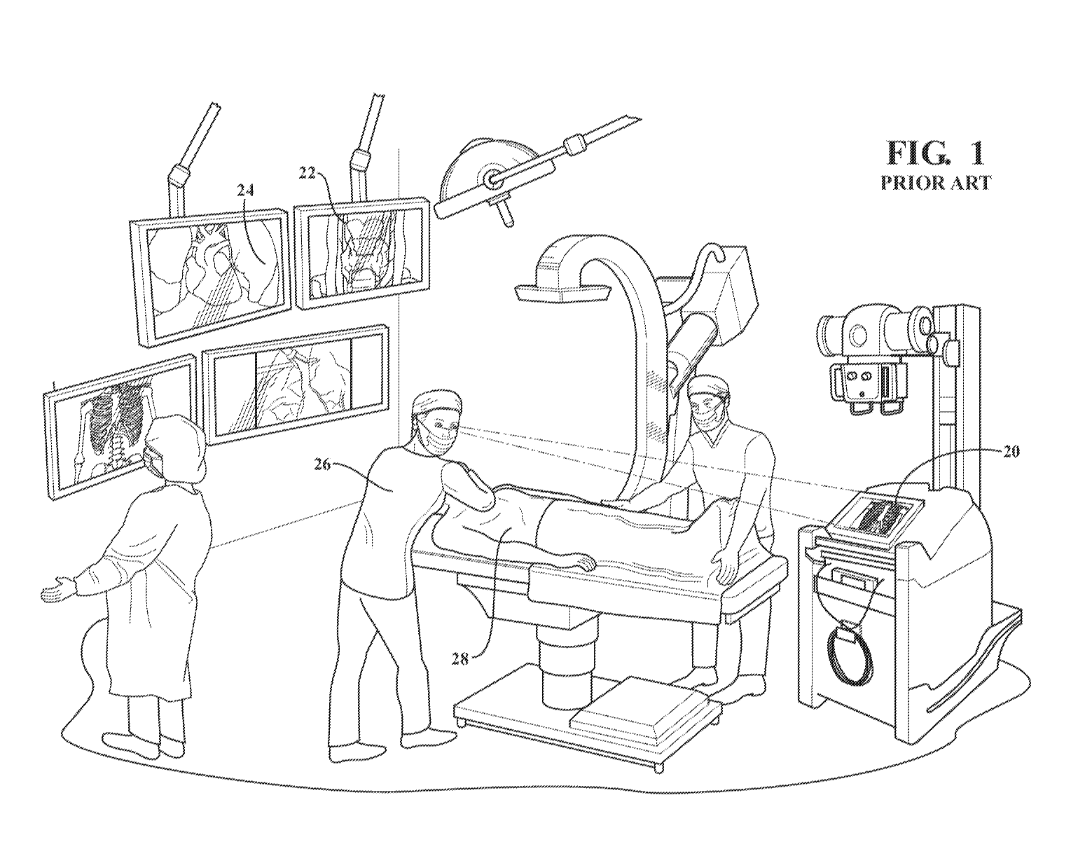

### DETAILED DESCRIPTION OF THE INVENTION

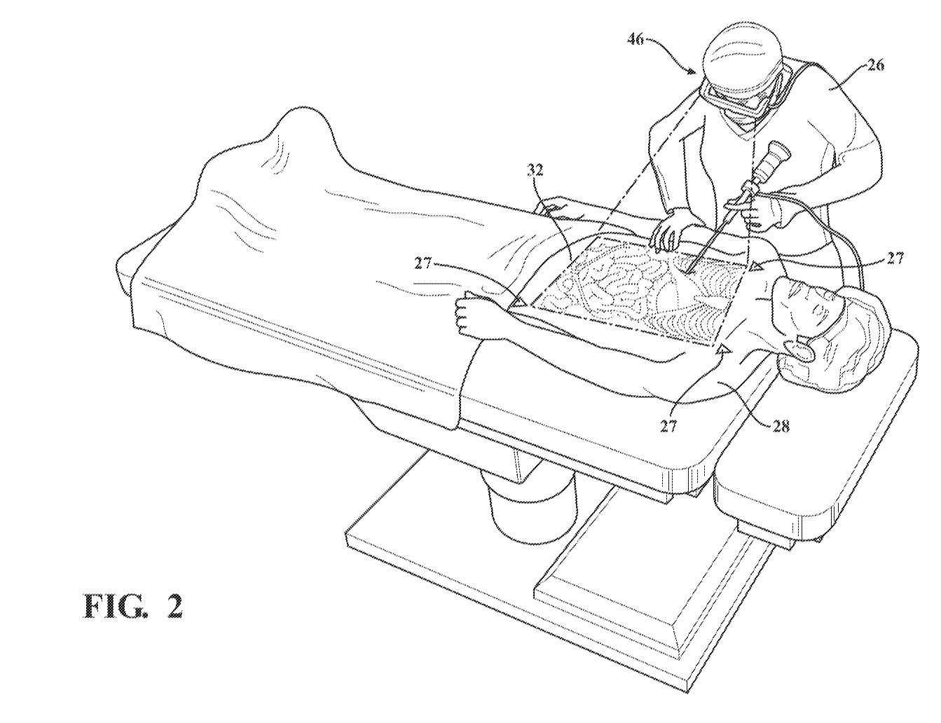

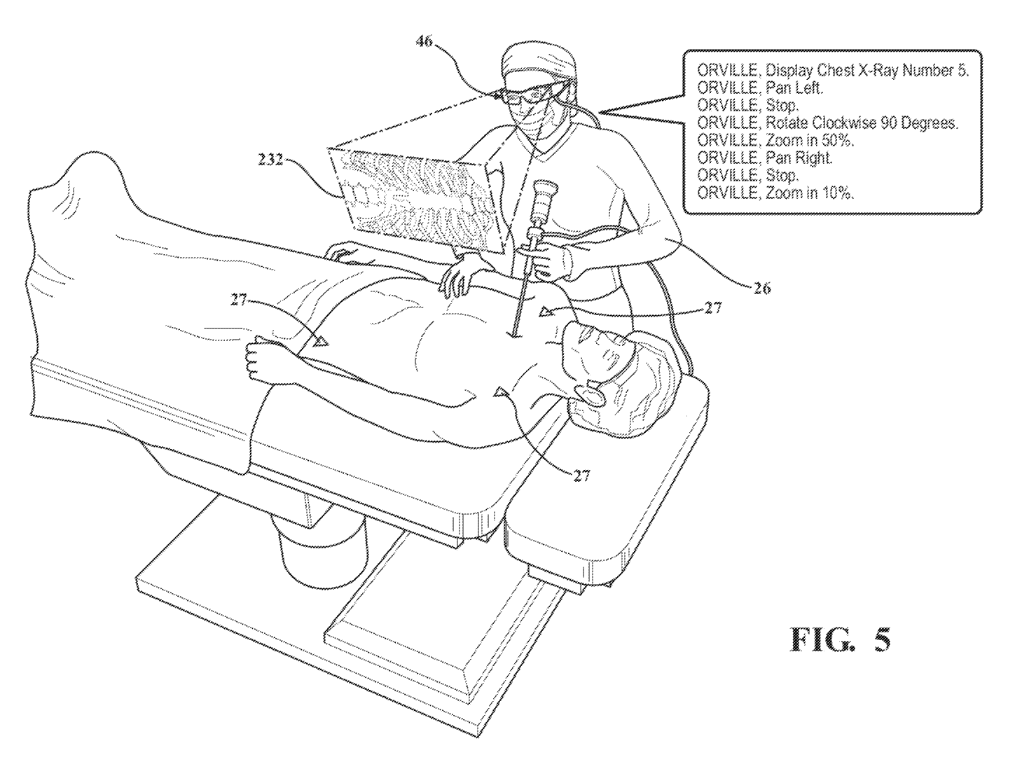

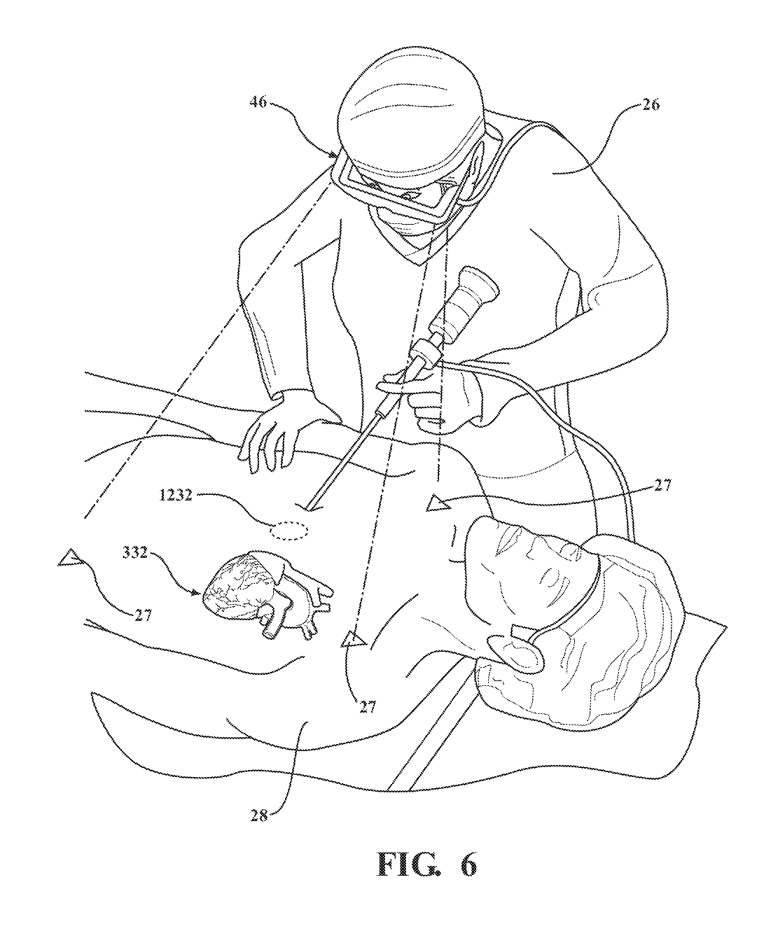

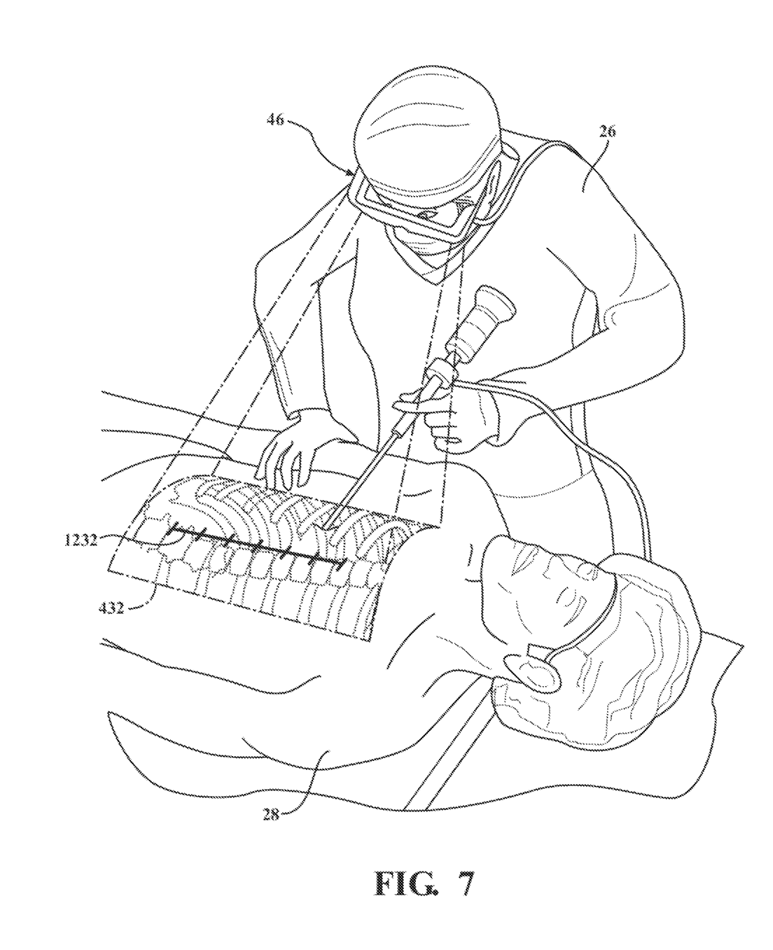

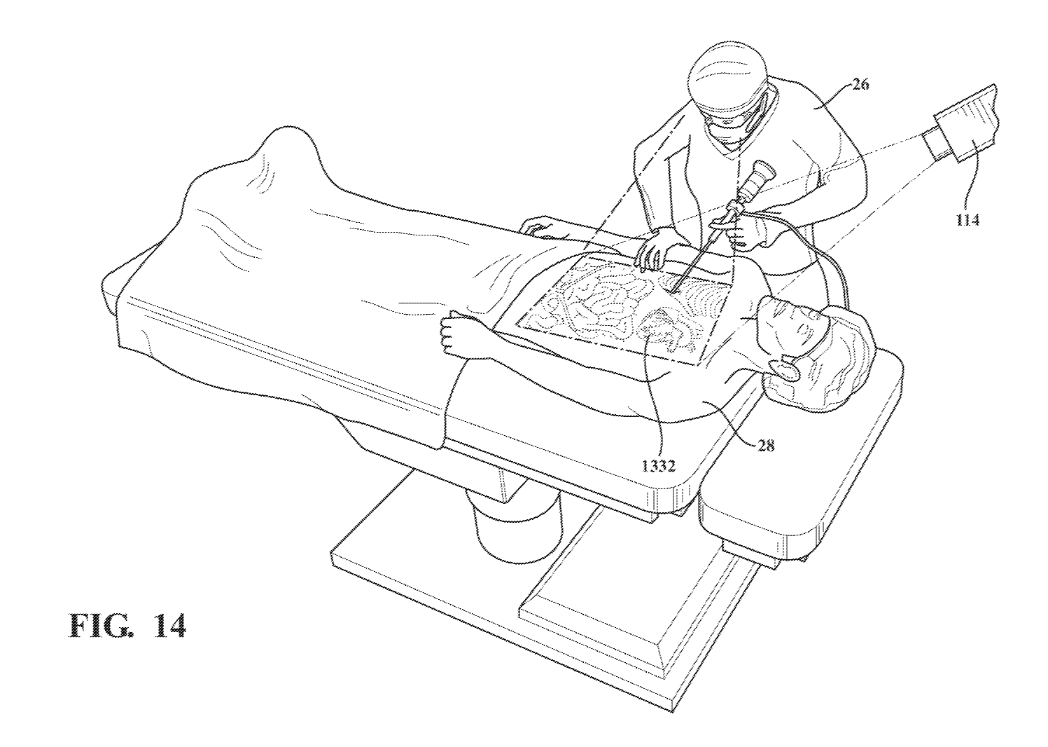

The exemplary embodiment can provide an intra-operative medical image viewing system 34 and method for displaying and interacting with two-dimensional, 2-½-Dimentional, or three-dimensional visual data in real-time and in perceived three-dimensional space. The system 34 can present a selectively or variably transparent image of an anatomical feature of a patient 28 to a surgeon 26 during surgery, as the surgeon 26 maintains a viewing perspective generally centered on the actual anatomical feature of the patient 28 or at least toward the patient 28 on whom some operation is being performed. The image as perceived by the surgeon 26 is selectively and/or variably transparent, in the sense that the surgeon 26 controls the image opacity throughout the range of fully transparent, e.g., when the image is not in use, to fully opaque, e.g., when high-contrast is desired, and through some if not all levels in-between. In most cases, the medical image appears to the surgeon to be located between herself, i.e., her eyes, and the patient 28. Typically, the image will appear to hover over (FIGS. 2, 12 and 13) or be overlaid on the skin of the patient 28 (FIGS. 7 and 14), or have the appearance of being inside the patient's body volume (FIG. 6). In other cases, the surgeon 26 may wish to locate the appearance of the image conveniently adjacent to the patient 28, such as hovering directly above them (FIG. 5). The present invention is better able to manage the multitude of medical images needed to be viewed by a surgeon during a procedure by positioning the medical image between herself and her patient. Such positioning of the perceived appearance of the medical images (i.e., as perceived by the surgeon 26) can be accomplished via numerous techniques, including wearable devices, heads-up/teleprompter type devices, and projection devices. Any one or all of these device types, as well as any other suitable means, can be used to apply the concepts of this invention so that the medical image is positioned between the surgeon and her patient, or at least in a convenient adjacent location, so that a surgeon is not required to look away from the patient, so that the surgeon does not have to sustain heavy cognitive loading in order to mentally register all of exhibited images, and so that the surgeon does not suffer unnecessary additional physical stresses. It is noted that the term “surgeon” is not used in a limiting sense; the invention is not limited to systems that can only be used by a surgeon. It is also noted that patient data can be stored in an “upstream” image file and remain unchanged while a “downstream” image that is generated based on the image file is modified and manipulated. It is noted that while a human patient is illustrated in the Figures, one or more embodiments of the invention may be utilized in teaching or simulation environments, and/or in the care of a non-human.

The exemplary embodiment can provide an intra-operative medical image viewing system 34 and method that allows the surgeon to self-manage the vital medical images she may wish to reference during a surgical procedure so that the instances in which her attention is shifted away from the patient are reduced, so that she can reduce the cognitive loading associated with mentally registering all of the displayed images, and so that she will suffer less physical stresses on her body. During surgery, the surgeon 26 can use the intra-operative medical image viewing system to self-modify the image as desired and on-the-fly.

More specifically, the problem of distraction is attenuated by the present invention in that the images, as perceived by the surgeon, appear to overlay or hover in close proximity to the patient. As a direct result, the surgeon 26 will not need to frequently look away from her patient in order to see the desired images. A substantial benefit of mitigating distraction is that the risk of unwanted hand movements will decrease, and surgical accuracy will increase, when the surgeon is no longer required to turn her head to see important anatomical or pathological images. Additionally, cognitive load/cognitive distraction away from the surgical task can accumulate into increased productive surgical time and reduced (or even adverse) patient outcomes. Another potential benefit is reduced operating time, which may improve patient outcomes.

The problem of excessive cognitive loading may also be mitigated by the present invention through its ability to position and scale a medical image relative to the patient 28 from the perspective of the surgeon 26. That is to say, the present invention manipulates the way a medical image is exhibited so that it conforms to the surgeon's visual perspective. As a result, the surgeon 26 does not need to mentally correlate each medical image to her actual, natural view of the patient 28. In situations where a given medical image was taken (or is being rendered) from a perspective different than the instantaneous visual perspective of the surgeon 26, the invention adapts the presentation of the image (but not the image source data) through actions like panning, zooming, rotating and tilting, to better align with the patient thereby reducing the cognitive effort expended by the surgeon to make thoughtful use of the medical image. Considering the large number of medical images typically referenced by a surgeon during a medical procedure, the cumulative cognitive loading imposed on a surgeon will be greatly reduced and with it the mental fatigue will also be reduced.

The system 34 can reduce physical demands on the surgeon 26 by placing the medical images over the patient 28, or in some embodiments the image will appear directly adjacent the patient 28 in a hovering manner. By strategically placing medical images over or directly adjacent the patient 28, as perceived by the surgeon 26, the need for the surgeon 26 to frequently look away during surgery is substantially if not completely eliminated. As a result, the physical stresses of muscle, joint and eye strains will be mitigated. A surgeon using the present invention may experience a marked reduction in physical fatigue, thereby enabling her to perform at optimum ability during long shifts. Over time, the surgeon will be exposed to fewer workplace-related injuries thereby favorably extending her service career. In addition, a reduction in surgery time can directly benefit the patent and improve safety. In particular, faster surgical procedures mean reduced affects associated with anesthesia, reduced risk for infections, shorter hospital stays, reduced medical costs, and the like.

The present invention will enjoy accelerated adoption in the medical field by overcoming the natural barriers associated with the stereotypical resistance to complicated technologies by surgeons by and large. This natural market resistance is addressed in the present invention by enabling the surgeon 26 to choose how to communicate image control inputs to the system from among many different user-interface modalities. Regardless of which user-interface modality the surgeon 26 selects, each image control input implements a desire by the surgeon 26 to modify the displayed image so that the position, pose, orientation, scale, and spatial (3D) structure of the image is adaptively changed in real-time and overlaid on the surgeon's view. The system can thus allow the surgeon 26 to communicate image control inputs in any of a plurality of different user-interface modalities. Each user-interface modality represents a different communication medium or command language, such as voice, touch, gesture, etc. Accordingly, the system 34 can be more intuitive for the surgeon 26 to use because the surgeon can choose the user-interface modality that is most intuitive to her. Said another way, the plurality of user-interface modalities allows the surgeon 26 to interact with the system in the most comfortable manner to her, thereby obviating the need for the surgeon 26 to learn and/or maintain knowledge of just one particular user-interface modality. During surgery, the surgeon 26 can be freed to communicate with the system in the way most “natural” to the surgeon 26. As a result, the likelihood of ready adoption for this technology within the surgical field will be greatly increased.

The exemplary embodiment can provide an intra-operative medical image viewing system 34 that increases the available viewing options for a surgeon 26 by providing the surgeon 26 with various approaches to three-dimensional viewing. As will be described in greater detail below, three-dimensional images can be defined in different formats. One surgeon 26 may find three-dimensional images in one particular format useful while another surgeon 26 may prefer images in a different format. The system 34 can allow the surgeon 26 to choose the format in which three-dimensional images are displayed so that the information contained in the medical image will be most useful to the surgeon 26 at the particular moment needed and for a particular surgical procedure.

The exemplary embodiment can provide an intra-operative medical image viewing system 34 that maintains the registration of an image to an actual anatomical feature of the patient 28 despite head movement by the surgeon 26. The system 34 can allow the surgeon 26 to selectively register, i.e., lock, an image to an actual anatomical feature of the patient 28 or to some other fiducial marker associated with the patient 28. For example, the image can be overlaid on the patient's actual anatomical feature and, by using commands in a selected user-interface modality, the image can be sized to match the actual anatomical feature, thus creating the visual impression of a “true registration” and a form of augmented reality. And so, just as the patient 28 lies immobile even while the surgeon 26 moves her eyes and head, so too does the medical image appear to remain immobile, registered to the patient. In this context, the actual patient 28 can be the reference or source image, and the image of the anatomical or pathological feature can be the image that is aligned to the actual patient 28. Initial placement of an image in preparation for registration can be established by the surgeon 26 communicating image control inputs to the system, resulting in image changes such as positioning, scaling, rotating, panning, tilting, and cropping. Alternatively, the system 34 can be configured to automatically present a true registration, or registration at a predetermined hovering distance, such as by calibrating to one or more strategically arranged markers or fiducials 27 placed directly onto the body of the patient 28, as suggested in FIGS. 2, 5 and 6. Another form of image registration results when the surgeon 26 issues commands (in the selected user-interface modality) to position the image in some convenient location but not-aligned with the anatomical features of the patient 28. The surgeon 26 may wish the positioned image to remain locked in space, as it were, despite movements of her eyes or head. Thus, the surgeon 26 can also choose to register the appearance of the image relative to the patient 28 wherein the position of the image is (and may intentionally be) not precisely aligned with the actual anatomical feature and/or size of the image is not generally the same as the size of the actual anatomical feature (as perceived by the surgeon 26). After establishing an initial registration that is desired by the surgeon 26, the system 34 can monitor the movement of the surgeon 26 and change the image displayed to the surgeon 26 so that the initial registration can be maintained. To maintain the perception of image registration while the surgeon 26 is moving, the system 34 may incrementally change the position, scale, orientation, pose, and special structure of the image in real-time. Registration may require precise alignment of images taken in different modality. For example, an intra-operative object to be viewed beforehand, or pre-operative image taken by an x-ray scanner of the analytical part with a camera positioned adjacent to the surgeon's eyes. Landmarks in the x-ray image would correlate to bone structure, whereas landmarks with visual image correspond to flesh and structure of the anatomical part. Precise alignment of these landmarks subject to the variations described before requires the use of sophisticated mathematical techniques that rely on features, fiducial information, image distance, and the like. Thus, the surgeon 26 can move more intuitively during the surgical procedure without concern for upsetting the initial image registration.

FIG. 2 is a perspective view of one embodiment of the invention shown in a first surgical environment. The surgeon 26 can be operating on the patient 28. The surgeon 26 can be wearing a display 30 suitable for implementing an intra-operative medical image viewing system 34 according to this invention. The intra-operative medical image viewing system 34 can allow the surgeon 26 to maintain a viewing perspective on the patient 28 while concurrently obtaining relevant image-based (i.e., pictorial) information about the patient 28 on-demand or on-the-fly. The display 30 can be positionable between the surgeon 26 and the patient 28 during surgery. The display 30 can be selectively and/or variably transparent and configured to exhibit at least one medical image 32 to the surgeon 26 that is overlaid on the patient 28 or that is positioned in an adjacent hovering location as perceived by the surgeon 26. In one embodiment of this invention, the display 30 can be a goggle-type system worn by the surgeon 26. As but one example, an Epson® Moverio® BT-200 can be utilized as the display 30. In one or more other embodiments of the invention, the display 30 can instead be mounted on a frame between the surgeon 26 and the patient 28, in the nature of a window or a “windshield.” In yet another example, the display 30 can be a more akin to a teleprompter-type screen device that can be placed over or above the patient 28. A further example embodies the invention as a projector, displaying imagery directly on the patient, as in FIG. 14.

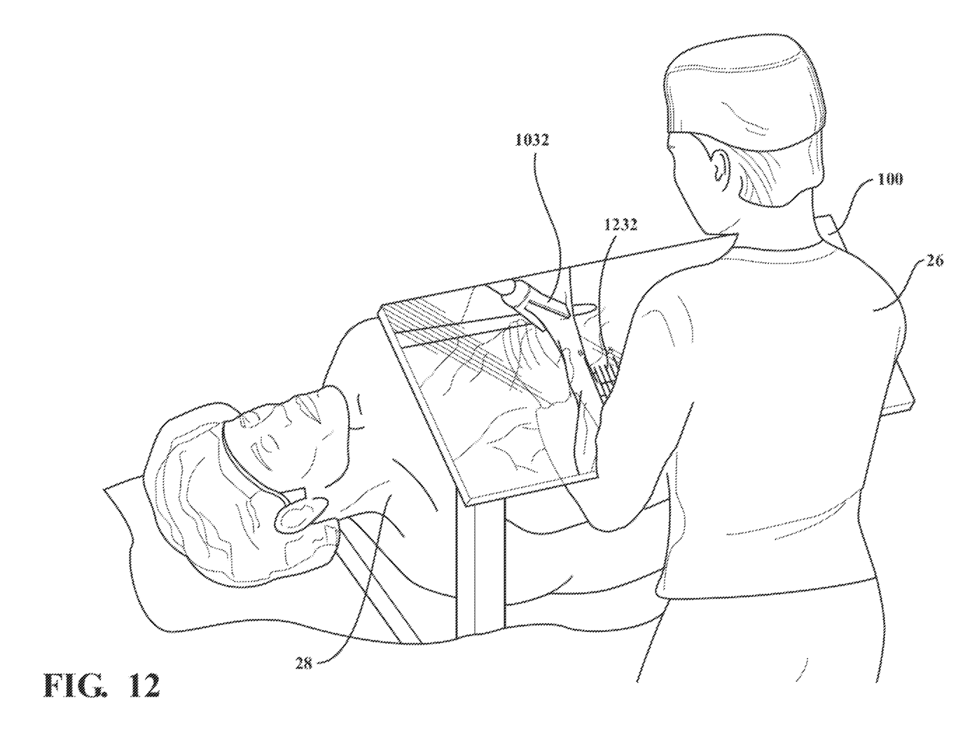

The image 32 can be two-dimensional and, as perceived by the surgeon 26, overlaid on the patient 28. The image 32 can preferably be a visual representation of an anatomical feature of the patient 28, however in other embodiments the image could be a graphical or numerical read-out or a measurement scale as in FIG. 12. In FIG. 2, the exemplary image 32 is suggested as an x-ray of the chest of the patient 28, but of course any type of digital medical image is possible. FIG. 2 illustrates how the image 32 can be perceived by the surgeon 26 as hovering directly above the body of the patient 28. In other embodiments, the image 32 may appear (to the surgeon 26) to be projected directed onto the body surface of the patient 28 or projected inside the patient's body.

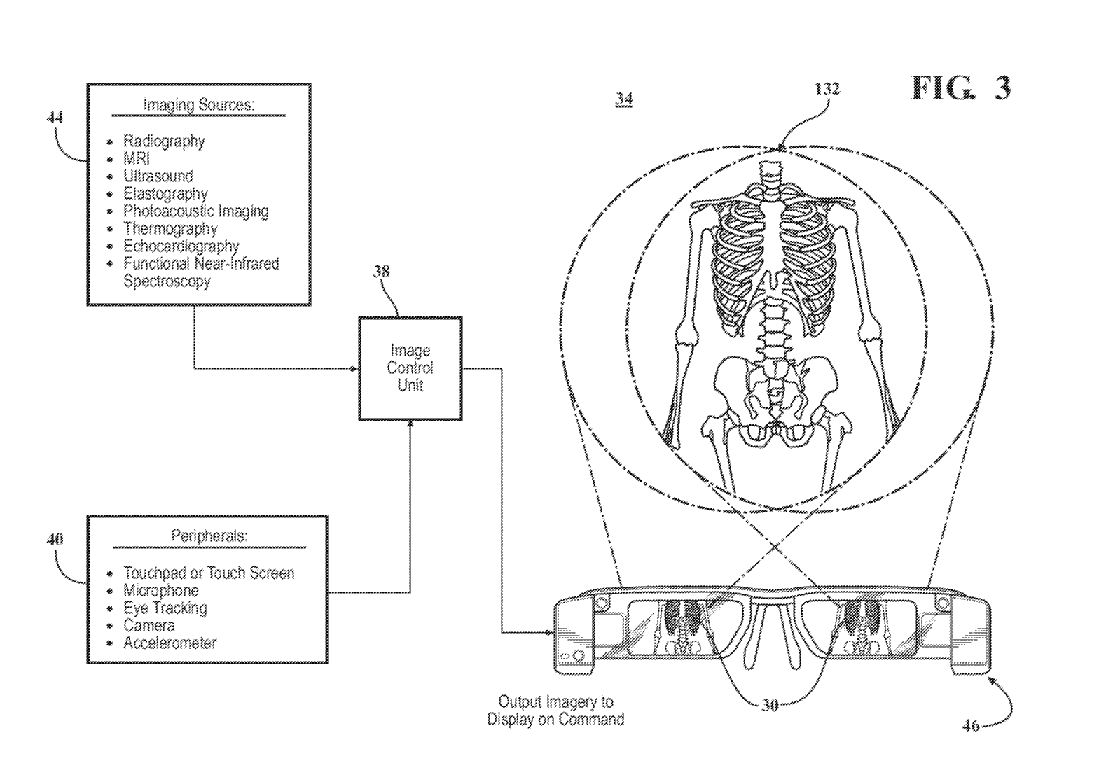

FIG. 3 is a schematic view of the embodiment of the invention in a second surgical environment. The intra-operative medical image viewing system 34 can include a plurality of image sources 44. Each image source 44 can have at least one digital image file representative of an anatomical or pathological feature of a patient 28. An image file can be of static data such as a picture or an x-ray or can be dynamic such as a video feed. In the latter example of a video feed, the image source 44 might produce digital images for an anatomical or pathological feature of the patient 28 in the form of a live data stream. In practice, it is likely each image source 44 will have many digital image files of the patient 28. An exemplary list of some of the many possible image sources 44 is identified in FIG. 3 by the general nature of the images retained. By way of example and not limitation, the system 34 can utilize images generated by radiography, computer-aided tomography, positron emission tomography, single-phase emission tomography, magnetic resonance imaging (MRI), ultrasound, elastography, photo-acoustic imaging, thermography, echocardiography, functional near-infrared, and spectroscopy. Each image source 44 can be a collection (or archive or database) of previously-created digital images, both pre-operative and intra-operative, or can be a source of real-time digital images.

The intra-operative medical image viewing system 34 can also include an image control unit 38 configured to retrieve the image file from the image source 44 and control the display 30 to exhibit (i.e., to display or render) and also to modify at least a portion of the at least one image 132. The at least one image 132 can be stored in the form of an image file. Modifying the way the at least one image 132 is displayed to the surgeon need not modify the image file itself. (The reference number for the image 132 in FIG. 3 is offset by one hundred to signify that is has been rendered from a different image file from that of FIG. 2.) The image control unit 38 can be configured to control the display 30 to modify the image 132 overlaid on the patient 28 by at least one of panning, zooming and rotating the image 132, as well as tilting, key-stoning, half-toning, texturing, wrapping or other image manipulation techniques. It should again be noted that the image control unit 38 only adapts the depiction of the image as it is perceived by the surgeon 26, and does not modify the source data in the image file.

The intra-operative medical image viewing system 34 can also include a plurality of peripheral devices 40, or as sometimes simply called peripherals. Each peripheral device 40 can be configured to receive an image control input from the surgeon 26. By way of example and not limitation, a peripheral 40 applied in one or more embodiments of the invention can be a microphone, a camera, an eye tracker, a mouse, a touch screen, and/or an accelerometer. By way of example and not limitation, an image control input can be the voice of the surgeon 26 communicated through the microphone, a hand gesture executed by the surgeon 26 and captured by the camera or motion-capture sensor, eye movements by the surgeon 26 detected by the eye tracker, the movement of the mouse by the surgeon 26, the touch of the surgeon 26 applied to the touch screen, or a nod of the head of the surgeon 26 detected by the accelerometer, or a body movement sensed by any suitable type of sensing equipment.

In response to the image control input by the surgeon 26, the respective peripheral 40 generates an image control signal. The image control input can be representative of a desire by the surgeon 26 to modify the image 132 exhibited by the display 30. The image control input signal can be a digital or analog signal. Each of the plurality of peripheral devices 40 defines a different user-interface modality for communicating a desire of the surgeon 26 to manipulate the image 132. By way of example and not limitation, a user-interface modality can be sound such as communicated through a microphone, body-motion in free space such as a hand gesture executed by the surgeon 26 and captured by a sensor or a camera, or eye movements by the surgeon 26 detected by an eye tracker, or physical movement of an object such as the movement of a joystick or computer mouse by the surgeon 26, or a measured movement of the head of the surgeon 26 detected by the accelerometer, or proximity/physical contact such as the touch of the surgeon 26 applied to a touch screen device. These are but a few of the many possible forms of user-interface modalities.

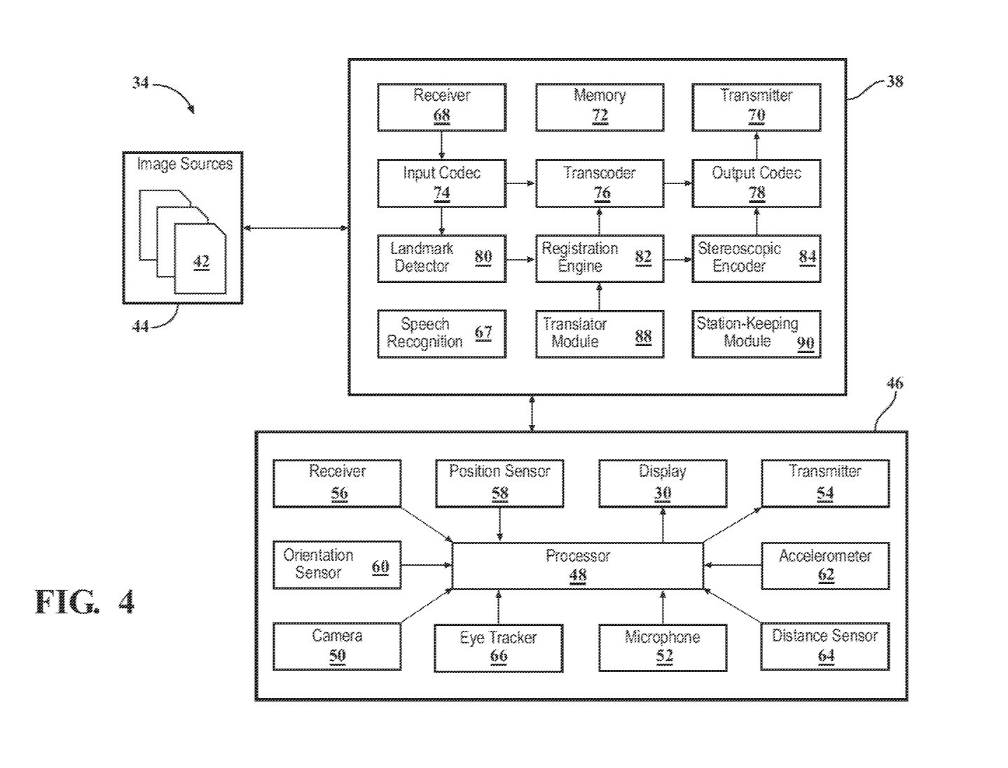

FIG. 4 is another schematic view of the embodiment of the invention. The intra-operative medical image viewing system 34 can also include the image control unit 38 configured to retrieve an image file 42 from an image source 44 and control the display 30 to exhibit and modify an image, such as image 32 or image 132 which are shown in previous Figures. The intra-operative medical image viewing system 34 can also include a plurality of peripheral devices 40. The peripheral devices 40 can be distinct from or integral with the display 30. The display 30 can be a component of a head mountable unit 46, such as the above-mentioned Epson® Moverio® BT-200. The head mountable unit 46 can thus be worn by the surgeon 26 while the surgeon 26 is operating on the patient 28.

The head mountable unit 46 can include a processor 48, one or more cameras 50, a microphone 52, the display 30, a transmitter 54, a receiver 56, a position sensor 58, an orientation sensor 60, an accelerometer 62, an all-off or “kill switch,” and a distance sensor 64, to name but a few of the many possible components. The processor 48 can be operable to receive signals generated by the other components of the head mountable unit 46. The processor 48 can be operable to control the other components of the head mountable unit 46. The processor 48 can be operable to process signals received by the head mountable unit 46. While one processor 48 is illustrated, it should be appreciated that the term “processor” can include two or more processors that operate in an individual or distributed manner.

The head mountable unit 46 can include one or more cameras, such as camera 50 and camera (or eye tracker) 66. Each camera 50, 66 can be configured to generate a streaming image or video signal. The camera 50 can be oriented to generate a video signal that approximates the field of view of the surgeon 26 wearing the head mountable unit 46. Each camera 50, 66 can be operable to capture single images and/or video and to generate a video signal based thereon.

In some embodiments of the disclosure, camera 50 can include a plurality of forward-facing cameras and position and orientation sensors. In such embodiments, the orientation of the cameras and sensors can be known and the respective video signals can be processed to triangulate an object with both video signals. This processing can be applied to determine the distance and position of the surgeon 26 relative to the patient 28. Determining the distance that the surgeon 26 is spaced from the patient 28 can be executed by the processor 48 using known distance calculation techniques. A plurality of position and orientation inputs could come from cameras, accelerometers, gyroscopes, external sensors, forward-facing cameras pointed at fiducials on the patient, and stationary cameras pointed at the surgeon 26.

Processing of the one or more forward-facing video signals can also be applied to determine the identity of the object. Determining the identity of the object, such as the identity of an anatomical or landmark feature of the patient 28, can be executed by the processor 48. Forward-facing cameras may stream image data for pattern-recognition logic to determine anatomical features, or they may simply look for one or more fiducial markers in the patient field and use those for alignment. A fiducial could be an anatomical feature, but it is more commonly a marker that has been placed in the visual field, for orientation reference. As will be discussed below, the image control unit 38 can also be configured to determine the identity of an object within the field of view of the surgeon 26. If the processing is executed by the image control unit 38, the processor 48 can modify the video signals to limit the transmission of data back to the image control unit 38. For example, the video signal can be parsed and one or more image files can be transmitted to the image control unit 38 instead of a live video feed.

The eye tracker or camera 66 can include one or more inwardly-facing cameras directed toward the eyes of the surgeon 26. A video signal revealing the eyes of the surgeon 26 can be processed using eye tracking techniques to determine the direction that the surgeon 26 is viewing. In one example, a video signal from an inwardly-facing camera can be correlated with one or more forward-facing video signals to determine the object the consumer is viewing. Further, the video captured by the camera 66 can be processed by the processor 48 or image control unit 38 to determine if the surgeon 26 has intentionally generated an image control input, such as by blinking in a predetermined sequence or glancing in a certain direction.

The microphone 52 can be configured to capture an audio input that corresponds to sound generated by and/or proximate to the surgeon 26. The audio input can be processed by the processor 48 or by the image control unit 38. For example, verbal inputs can be processed by the image control unit 38 such as “pan left,” “zoom,” and/or “stop.” The processor 48 or the image control unit 38 can include a speech recognition module 67 to implement known speech recognition techniques to identify speech in the audio input. (In FIG. 4, the speech recognition module 67 is shown only in the one example as part of the image control unit 38, it being understood that an alternative arrangement could associate the speech recognition module 67 with the processor 48.) Such audio inputs can be correlated to the video inputs generated by the camera 50 in order to register an image with an anatomical feature of the patient 28. Some surgeons are accustomed to giving verbal commands, whereas others will prefer other user-interface modalities to control the images displayed; the invention does not impose one particular user-interface modality.

The display 30 can be preferably positioned within the field of view of the surgeon 26. Video content called-up on-demand by the surgeon 26 can be shown to the surgeon 26 with the display 30. The display 30 can be configured to display text, graphics, images, illustrations and any other video signals to the surgeon 26. The display 30 may be almost fully transparent when not in use, and remain partially transparent when in use to minimize the obstruction to the surgeon 26 of the field of view through the display 30. Preferably, the degree of transparency is variable throughout the range from fully transparent to fully opaque. In some situations, the surgeon may prefer full opacity, such as for example in the case of a black-and-white CT scan where high-contrast is beneficial. An all-off or “kill switch” can be integrated into the display 30 to cause the image to turn off or render transparent after a predetermined period of inactivity. The display 30 can be configured for toggleable transparency, and variable along the spectrum from full opaque to full transparency, depending on user preference.

The transmitter 54 can be configured to transmit signals, commands, or control signals generated by the other components of the head mountable unit 46 over a plurality of communications media, wired or wireless. The processor 48 can direct signals to the head mountable unit 46 through the transmitter 54. The transmitter 54 can be an electrical communication element within the processor 48. In one example, the processor 48 can be operable to direct the video and audio signals to the transmitter 54, and the transmitter 54 can be operable to transmit the video signal and/or audio signal from the head mountable unit 46, such as to the image control unit 38.

The head mountable unit 46 and image control unit 38 can communicate by wire or through a network 20. As used herein, the term “network” can include, but is not limited to, a Local Area Network (LAN), a Metropolitan Area Network (MAN), a Wide Area Network (WAN), the Internet, an Internet of Things or combinations thereof. Embodiments of the present disclosure can be practiced with a wireless network, a hard-wired network, or any combination thereof.

The receiver 56 can be configured to receive signals and direct signals that are received to the processor 48 for further processing. The receiver 56 can be operable to receive transmissions from the network and then communicate the transmissions to the processor 48. The receiver 56 can be an electrical communication element within the processor 48. In some embodiments, the receiver 56 and the transmitter 54 can be an integral unit.

The transmitter 54 and receiver 56 can communicate over a Wi-Fi network, allowing the head mountable unit 46 to exchange control signals wirelessly (using radio waves or other types of signals) over a computer network, including point-to-point connections or high-speed Internet connections. The transmitter 54 and receiver 56 can also apply Bluetooth® standards for exchanging control signals over short distances by using short-wavelength radio transmissions, and thus create a personal area network (PAN). The transmitter 54 and receiver 56 can also apply 3G or 4G (or higher as the available technology permits), which is defined by the International Mobile Telecommunications-2000 (IMT-2000) specifications promulgated by the International Telecommunication Union.

The position sensor 58 can be configured to generate a position signal indicative of the position of the head of the surgeon 26 within the surgical field and/or relative to the patient 28. The position sensor 58 can be configured to detect an absolute or relative position of the surgeon 26 wearing the head mountable unit 46. The position sensor 58 can electrically communicate a position signal containing a position control signal to the processor 48 and the processor 48 can control the transmitter 54 to transmit the position signal to the image control unit 38 through the network. Identifying the position of the head of the surgeon 26 can be accomplished by radio, ultrasound or ultrasonic, infrared sensors, visible-light cameras, or any combination thereof. The position sensor 58 can be a component of a real-time locating system, which can be used to identify the location of objects and people in real time within a building such as a hospital. The position sensor 58 can include a tag that communicates with fixed reference points in the operating room, on the patient, or the hospital or care facility. The fixed reference points can receive wireless signals from the position sensor 58.

The orientation sensor 60 can be configured to generate an orientation signal indicative of the orientation of the head of the surgeon 26, such as the extent to which the surgeon 26 is looking downward, upward, or parallel to the ground. A gyroscope can be a component of the orientation sensor 60. The orientation sensor 60 can generate the orientation signal in response to the orientation that is detected and communicate the orientation signal to the processor 48.

The accelerometer 62 can be configured to generate an acceleration signal indicative of the motion of the surgeon 26. The accelerometer 62 can be a single axis or multi-axis accelerometer. The orientation sensor 60 could thus be embodied by a multi-axis accelerometer. The acceleration signal can be processed to assist in determining if the surgeon 26 has moved or nodded.

The distance sensor 64 can be operable to detect a distance between an object and the head mountable unit 46. The distance sensor 64 can be operable to detect the presence of anatomical features of the patient 28 without any physical contact. The distance sensor 64 can detect changes in an electromagnetic, visible, or infrared field. Alternatively, the distance sensor 64 can apply capacitive photoelectric principles or induction. The distance sensor 64 can generate a distance signal and communicate the distance signal to the processor 48. The distance signal can be used to determine movements of the surgeon 26. The distance signal can also be useful when processed with video signals to recognize or identify the anatomical feature being observed by the surgeon 26.

The image control unit 38 can include one or more processors and can define different functional modules including a receiver 68, a transmitter 70, memory 72, an input codec 74, a transcoder 76, an output codec 78, a landmark detector 80, a registration engine 82, a stereoscopic encoder 84, a translator module 88, and a station-keeping module 90. The receiver 68 can be configured to receive signals and direct signals that are received to the other modules of the image control unit 38 for further processing. The receiver 68 can be operable to receive transmissions from the network. In some embodiments of the present disclosure, the receiver 68 and the transmitter 70 can be an integral unit.

The transmitter 70 can be configured to transmit signals, commands, or control signals generated by the other components of the image control unit 38. The image control unit 38 can direct signals to the head mountable unit 46 through the transmitter 70. The image control unit 38 can be operable to direct the video signal to the transmitter 70 and the transmitter 70 can be operable to transmit the video signals from the image control unit 38, such as to the head mountable unit 46.

The transmitter 70 and receiver 68 can communicate over a Wi-Fi network, allowing the image control unit 38 to exchange control signals wirelessly (e.g., using radio waves) over a computer network, including point-to-point connections or high-speed Internet connections. The transmitter 70 and receiver 68 can also apply Bluetooth® standards for exchanging control signals over short distances by using short-wavelength radio transmissions, and thus create a personal area network (PAN). The transmitter 70 and receiver 68 can also apply 3G or 4G (or higher if available), which is defined by the International Mobile Telecommunications-2000 (IMT-2000) specifications promulgated by the International Telecommunication Union.

Memory 72 can be any suitable storage medium (flash, hard disk, etc.). System programming can be stored in and accessed from memory 72. Any combination of one or more computer-usable or computer-readable media may be utilized in various embodiments of the invention. For example, a computer-readable medium may include one or more of a portable computer diskette, a hard disk, a random access memory (RAM) device, a read-only memory (ROM) device, an erasable programmable read-only memory (EPROM or Flash memory) device, a portable compact disc read-only memory (CDROM), an optical storage device, and a magnetic storage device. Computer program code for carrying out operations of this invention may be written in any combination of one or more programming languages.

The input codec 74 can receive the image file 42 from the image source 44 and decompress the image file 42. If the image defined by the image file 42 is not to be modified or analyzed, the decompressed image file 42 can be transmitted to the transcoder 76. The transcoder 76 can convert to the image file 42 to a different format of similar or like quality to gain compatibility with another program or application, if necessary.

If the image defined by the image file 42 is be modified or analyzed, the decompressed image file 42 can be transmitted to landmark detector 80. For example, video signals generated by the camera 50 can be processed by the landmark detector 80 to identify an anatomical feature of the patient 28. The landmark detector 80 of the image control unit 38 can be configured to determine the identity of an object within the field of view of the surgeon 26. When the identity of an object within the field of view of the surgeon 26 is determined, the landmark detector 80 of the image control unit 38 can communicate the identity to the registration engine 82. The registration engine 82 can generate image modification commands that can be transmitted to the display 30 in order to register and overlay the image to the object on the display 30. The object can be an anatomical feature of the patient 28. For example, an image such as an x-ray of a vertebrae can be registered to the actual vertebrae of the patient 28 as viewed by the surgeon 26.

The camera 50, microphone 52, and camera 66 can define the peripheral devices 40 configured to receive an image control input from the surgeon 26. The surgeon 26 can use any of the peripheral devices 50, 52, 66 (or others) in one or more embodiments of the system 34 to communicate a desire to manipulate an image displayed by the display 30, without changing the underlying patient data stored in the image file. The surgeon 26 can use any of the peripheral devices 50, 52, 66 (or others) concurrently in one or more embodiments of the system 34 to communicate a desire to manipulate an image displayed by the display 30.

The translator module 88 can be configured to receive the image control signals from the plurality of peripheral devices in different user-interface modalities. The translator module 88 can be configured to convert the respective image control signals into an image manipulation command in a common operating room viewer language. For example, the surgeon 26 can say “zoom” or nod her head or open her hand to zoom in on the image exhibited by the display 30. The translator module 88 converts these various image control signals (generated from respective image control inputs) into the same image manipulation command.

The common operating room viewer language may, in some respects, be likened to the Musical Instrument Digital Interface, or MIDI. MIDI is a technical standard that describes a protocol, digital interface and connectors and can allow a wide variety of electronic musical instruments, computers and other related devices to connect and communicate with one another. The common operating room viewer language can function similarly. The system 34, through the common operating room viewer language, can allow the surgeon 26 to communicate image control inputs in any of a plurality of different user-interface modalities. Thus, the system 34 can allow the surgeon 26 to utilize the system 34 more intuitively and obviate the requirement that the surgeon 26 learn and maintain knowledge of one particular user-interface modality. During surgery, the surgeon 26 can be freed to communicate with the system 34 using one or more peripherals that are regarded as most “natural” to the surgeon 26. Thus, the surgeon 26 can be more focused on the patient and not on communicating properly with an image retrieval system using a frustrating modality. For example, if the surgeon 26 prefers using voice commands, the surgeon 26 will choose the peripheral that enables this type of image control inputs. Alternatively, if the surgeon 26 prefers using eye movement commands, the surgeon 26 will choose the peripheral that enables this type of image control inputs. And, if the surgeon 26 prefers using hand movement commands, the surgeon 26 will choose a peripheral that enables that type of image control inputs. And so forth.

The translator module 88 can transmit the image manipulation command to the registration engine 32. The registration engine 32 compiles and applies all of the image manipulation commands to be applied to the image. The registration engine 32 of the image control unit 38 controls the display 30 in response to the image manipulation commands received from the translator module 88.

The translator module 88 is illustrated in FIG. 4 as part of the image control unit 38. However, as shown in FIG. 3, the translator module 88 can be physically distinct from the image control unit 38. The system 34 can include a computing device comprising one or more processors and a non-transitory, computer readable medium, such as memory 72. It should be appreciated that a computing device can operate in a parallel or distributed architecture. Thus, the image control unit 38 and the translator module 88 can be physically distinct and can cooperatively define a single computing device according to the present invention.

In the embodiment of the invention shown in FIG. 4, image control signals from the various peripherals can be received by a common translator module 88. In the embodiment of the invention, at least one of the one or more processors of the computing device of the system 34 can be integral with each peripheral device 40. In other words, each peripheral 40 can include a respective translator module. The translator module of each peripheral 40 can be configured to convert the image control input into an image manipulation command in the common operating room viewer language and transmit the image manipulation command to another of the at least one of the one or more processors of the computing device.

FIG. 5 is a perspective view of the embodiment of the invention in a third surgical environment. FIG. 5 illustrates and example of the surgeon 26 using the microphone 52 as the preferred peripheral. The surgeon 26 can be speaking voice commands to modify the image 232. (The reference number for the image 232 in FIG. 5 is offset by two hundred to signify that is has been rendered from a different image file from that of the preceding figures.) In this embodiment, the surgeon 26 can speak the word “ORVILLE” to alert the system 34 that an image control input follows. Of course, the word “ORVILLE” can be offered merely as an example; in practice the system can be configured to respond to any suitable word or phrase or sound. The microphone 52 can convert the image control input, which in this case can be the captured voice of the surgeon 26, to an image control signal such as an analog signal, which can be converted by the translator module 88 into an image manipulation command.

The image can include portions indicating a three-dimensional nature of the anatomical feature of the patient 28. The image control unit 38 can be configured to modify an image in response to the image control signal in any one of a plurality of different two-dimensional, 2-½-dimensional, or three-dimensional modalities. FIG. 3 illustrates the application of a first three-dimensional modality, displaying a stereoscopic feed through a binocular viewer. The head mountable unit 46 displays the image 132 to the left and right eye such that the fields of view for each eye partially overlap to create binocular vision.

FIG. 6 illustrates the application of a second three-dimensional modality, “holographic 3D” in which three-dimensional tomography (or other suitable form) data can be used to create a feature-specific three-dimensional anatomical view. The surgeon 26 is shown performing a procedure on or near the heart of the patient 28. The image 332 displayed to the surgeon 26 appears to the surgeon 26 as three-dimensional. The image 332 can be an amalgam or fusion of several tomographic slices that have been stitched together so as to create a 3D image. (The reference number for the image 332 in FIG. 6 is offset by three hundred to signify that is has been rendered from a different image file from that of the preceding figures.) A treatment guide 1232 can be overlaid on (i.e., combined or rendered with) the image 332 that is visible to the surgeon 26 so that the two images 332, 1232 are aligned in true registry. In this example, the treatment guide 1232 represents a tumor boundary. However, the treatment guide 1232 could take many different forms including that of a scale (FIGS. 7 and 12), a radiologic study, pre-operative sketches or notes made by the surgeon 26 herself or perhaps by a teacher or a consulting practitioner or a medical student. For example, a radiologist may draw guiding lines or annotations pre-operatively for a surgeon to study. When a treatment guide 1232 is dimensionally relevant, e.g., in the case of a scale or tumor boundary, the two displayed images 332, 1232 will be rendered in full registry with one another so that any panning, zooming, rotating or tilting of the one image 332 will be accompanied by a corresponding manipulation of the other image 1232.

FIG. 7 illustrates the application of a third three-dimensional modality, sometimes referred to as “false 3D” or “2.5D” in which a two-dimensional image can be wrapped around a three dimensional structure, namely the body surface of the patient 28. The surgeon 26 is shown performing a procedure on the chest of the patient 28. The image 432 displayed to the surgeon 26 can be rendered by the system as having been wrapped over the chest of the patient 28 yielding the appearance of a three-dimensional image. (The reference number for the image 432 in FIG. 7 is offset by four hundred to signify that is has been rendered from a different image file from that of the preceding figures.) In this example, a treatment guide 1232 in the form of a measuring modality is applied together with the image 432. The treatment guide 1232 is depicted as a scale which could be provided for purposes of gauge and/or manipulation. As an example, an image-registered treatment guide 1232 in the form of a scale or gauge could be especially helpful in orthopedics procedures.

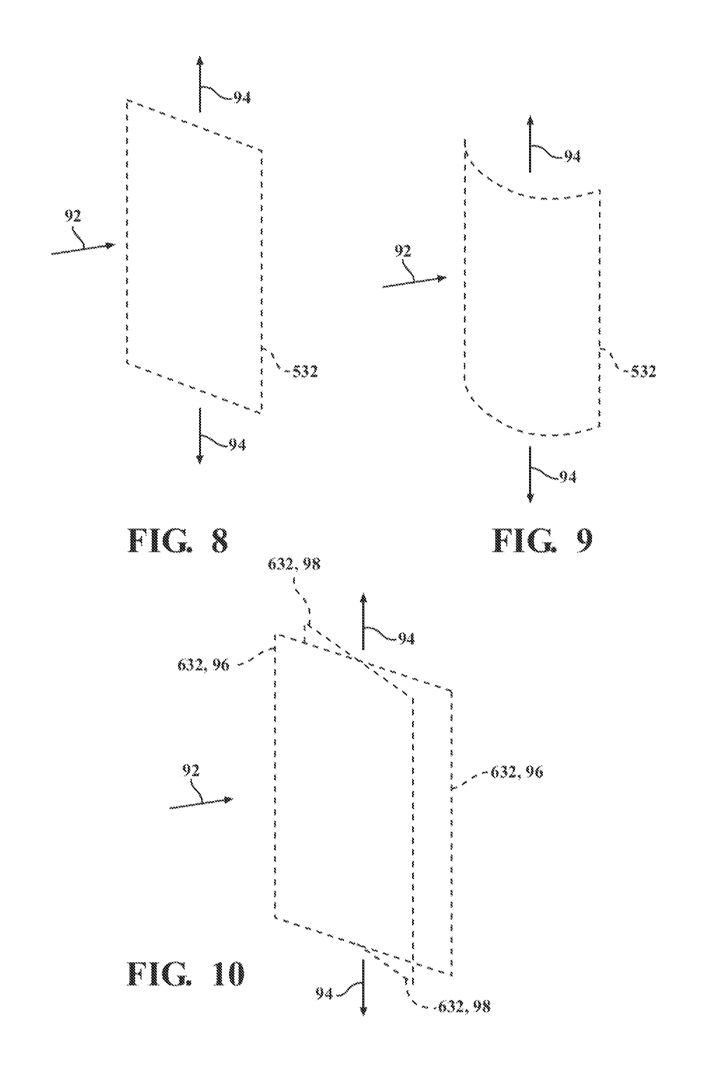

FIGS. 8 and 9 further illustrate the concept of image wrapping as introduced in the preceding FIG. 7. FIG. 8 is a perspective view of a two-dimensional image 532 in a planar configuration. The arrow referenced at 92 indicates the surgeon's viewing perspective. FIG. 9 is a perspective view of the two-dimensional image 532 shown in FIG. 8 but rendered in a warped configuration to mimic the surface curvature of the patient's body. In FIG. 9, the image 532 has been wrapped around the axis 94. (The reference number for the image 532 in FIGS. 8 and 9 is offset by five hundred to signify that is has been rendered from a different digital image file from that of the preceding figures.)

FIG. 10 illustrates the application of a fourth three-dimensional modality, “perspective 3D” in which an observable plane of a two-dimensional image can be changed to lend artificial depth. This is known as tilting or “key-stoning.” The arrow referenced at 92 indicates the viewing perspective. A two-dimensional image 632 is shown disposed in a first plane 96. The image 632 can be changed to a perspective 3D view by rotating the image 632 about the axis 94 to a plane 98. The viewing perspective 92 can be unchanged and thus the image 632 appears to have depth. (The reference number for the image 632 in FIG. 10 is offset by six hundred to signify that is has been rendered from a different image file from that of the preceding figures.)

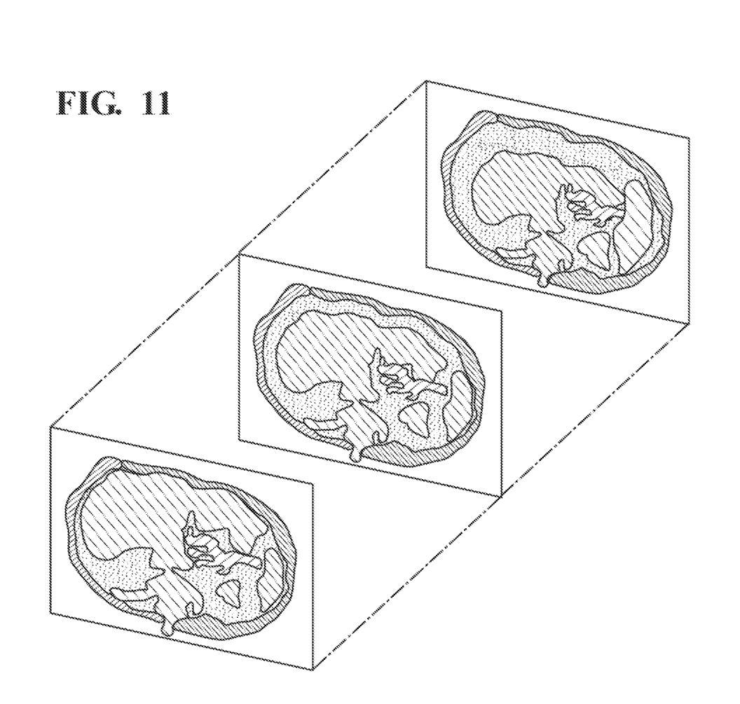

FIG. 11 illustrates the application of a fifth three-dimensional modality, “fly through 3D” in which a series of three-dimensional tomographic slices can be sequentially exhibited. Each tomographic slice can be a distinct image. The images allow a surgeon to gain an understanding of the patient's internal anatomy. As referred to previously, a fusion of several tomographic slices can be stitched together to create a 3D image.

FIG. 12 is a perspective view of the invention in a sixth surgical environment. Unlike previous embodiments, where the display was depicted as a wearable device, and in particular eye-ware, by the surgeon 26, the embodiment of FIG. 12 illustrates the example of a display 100 that is mounted on a frame and sized to be non-wearable by the surgeon 26. That is, the display 100 can be like a teleprompter screen or other see-through device that is capable of locating a medical image 1032 between the eyes of the surgeon 26 and the patient 28. The surgeon 26 is shown here holding a laparoscope. The intra-operative viewer 100 in this example is displaying imagery that is concurrently captured by a tiny camera inside the body of the patient 28 which is carried on the laparoscopic device. A treatment guide 1232, in the exemplary form of a scale, is shown overlaid on or combined with the laparoscopic imagery 1032 that is concurrently visible to the surgeon 26 so that the two images 1032, 1232 are aligned to one another in true registry. To achieve this registry, the system 34 can position, orient and/or pose one or both of the images 1032, 1232 as needed. It will be appreciated that in this example, the system 34 is employed to fuse together multiple images for the benefit of the surgeon 26. More specifically, two or more pre-operative and/or intra-operative images are fused together to present a “diagnostic” or “radiological”-like image to the surgeon 26. Once combined, the surgeon can then position and scale the combined medical images 1032, 1232 relative to the patient 28 so that the combined images 1032, 1232 conform to the surgeon's visual perspective. This will help the surgeon 26 further reduce the cognitive effort needed to make thoughtful use of the multiple medical images.

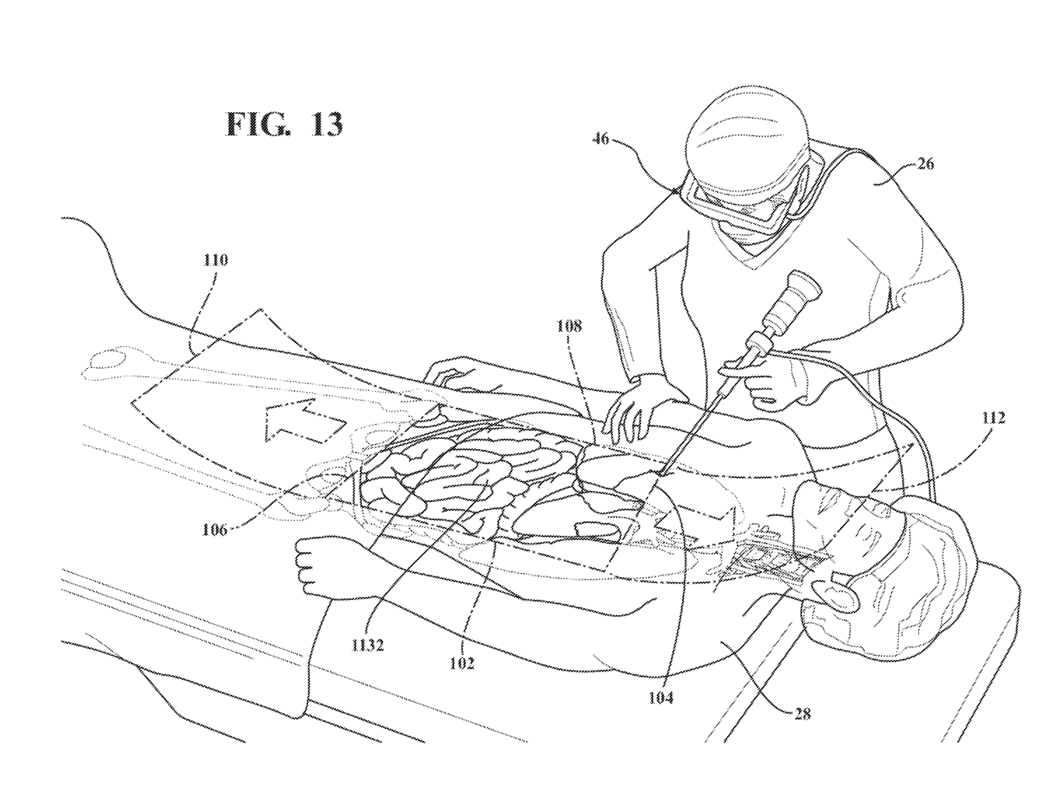

FIG. 13 is a perspective view of the invention in a seventh surgical environment. FIG. 13 illustrates the effect of the operation of the station-keeping module 90. The surgeon 26 is shown performing a procedure on the chest of the patient 28. From the visual perspective of the surgeon 26, an image 1132 from an image file associated with the patient 28 has been registered relative to the patient 28. The image 1132 could have been automatically registered or could have been registered in response to image control inputs generated by the surgeon 26. The image control unit 38 can be responsive to inputs from the surgeon 26 to modify the image 32 to allow the surgeon 26 to selectively position, size, alter pose, and orient the image 32 exhibited on the display 30. Such modifications of the image as perceived by the surgeon 26 may include, but are not limited to, selectively scaling, rotating, panning, tilting and cropping the image. Although not shown, a treatment guide could be overlaid on or combined with the image 1132 that is visible to the surgeon 26. The registered image 1132 thus defines a selected first configuration.

The image 1132 can be bound by edges lines 102, 104, 106, 108. If the surgeon 26 changes the direction of her viewing perspective, the image 1132 will accordingly change position on the display. For example, if the surgeon 26 looks toward the feet of the patient 28, the area bounded by the edge lines 102, 106, 108, 110, the image 1132 can disappear from the display. The structures shown in phantom and bounded by edge lines 102, 106, 108, 110 in FIG. 13 may or may not be presented on the display. Similarly, if the surgeon 26 looks toward the head of the patient 28, the area bounded by the edge lines 102, 104, 108, 112, the image 1132 can disappear from the display.

The station-keeping module 90 can include a position module, an orientation module, a registration module, a pan/tilt/zoom module, and an image recalibration module. The position module can be configured to detect a first position of the display 30 when the first configuration is selected and determine a change in position of the display 30 from the first position. The position module can receive signals from the position sensor 58 and process the signals to determine the position of the display 30. The orientation module can be configured to detect a first orientation of the display 30 when the first configuration is selected and determine a change in orientation of the display 30 from the first orientation. The orientation module can receive signals from the orientation sensor 60 (or a plurality of orientation sensors) and process the signals to determine the orientation of the display 30.

The registration module can be configured to determine a registration default condition defined by a frame of reference or a coordinate system. The first configuration of the image can be defined by the frame of reference or the coordinate system. The first position and the first orientation of the head of the surgeon 26 when the first configuration is selected can be defined by the frame of reference or the coordinate system. The frame of reference or coordinate system can be defined by fiducials or tags that communicate with fixed reference points.

The registration default condition can be representative of the field of view of the surgeon 26 when the first configuration is established. The image recalibration module can be configured to determine one or more image modification commands to be applied by the display 30 to change how the image should be displayed when the surgeon 26 has moved, i.e., when the forward field of view of the surgeon 26 has changed. The way the image is displayed can be changed by the station-keeping module 90 in response to eye movement, head movement and/or full body movement.

The image recalibration module can determine how the image should be changed. The changes to the image result in a second or modified image, which can be identified as a second configuration of the image. The image recalibration module can determine the attributes of the second configuration so that the second configuration is consistent with the registration default condition. With reference again to FIG. 6, if the surgeon 26 moves toward the feet of the patient 28, the second configuration of the image 332 can be of the downwardly facing edge of the heart of the patient 28. The registration default condition can be maintained by positioning image 332 at the same position in the field of view of the surgeon 26 as the first configuration. For example, the system 34 can keep the two-dimensional, 2-½-dimensional, or three-dimensional image 332 of the heart in place and the surgeon 26 could view the entire perimeter of the heart by walking around the patient while keeping her forward field of view in the direction of the fiducials or the area of the body of the patient 28 that was chosen for initial registration (such as the center of the chest).

In another example, with reference again to FIG. 5, if the surgeon 26 turns her head toward the head of the patient 28, the second configuration of the image 232 can be planar as can be the first configuration of the image 232 (shown in FIG. 5). However, the second configuration 232 but can be shifted to the right of the display 30 relative to the position of the first configuration of the image 232 on the display 30. Alternatively, if the surgeon 26 moves toward the feet of the patient 28, the observable plane of the second configuration of the image 232 can be shifted relative to the observable plane of the first configuration of the image 232. In other words, in this example, the observable plane of the first configuration of the image 232 can be similar to the plane 96 in FIG. 10 and the observable plane of the second configuration of the image 232 can be similar to the plane 98.

FIG. 14 is a perspective view of an embodiment of the invention in an eighth surgical environment. The surgeon 26 is shown performing a procedure on the chest of the patient 28. Unlike previous embodiments, where the display was depicted either as a wearable device or as a transparent teleprompter-like display screen, the embodiment of FIG. 14 illustrates the example of a display that is projected directly onto the surface of the patient 28. From the visual perspective to the surgeon 26, an image 1332 of the heart of the patient 28 can appear directly over and registered with the position of the actual heart of the patient. The image 1332 can be generated by a plurality of holographic projectors 114, 116, 118 positioned about the operating room. To restate, the perceived appearance of the medical images can be accomplished via any suitable device or technique. While examples have been provided of wearable devices, heads-up/teleprompter type devices, and projection devices, these are but a few examples. Indeed, any device capable of creating for the surgeon the perception of a medical image that is positioned between them and their patient, or at least in a convenient adjacent location, may be used to implement the teachings of this invention.

Example embodiments are provided so that this disclosure will be thorough, and will fully convey the invention scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known procedures, well-known device structures, and well-known technologies are not described in detail given the high level of ordinary skill in this art.

The terminology used herein is for the purpose of describing particular example embodiments only and is not intended to be limiting. As used herein, the singular forms “a,” “an,” and “the” may be intended to include the plural forms as well, unless the context clearly indicates otherwise. The term “and/or” includes any and all combinations of one or more of the associated listed items. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring her performance in the particular order discussed or illustrated, unless specifically identified as an order of performance. It is also to be understood that additional or alternative steps may be employed.

Unless specifically stated otherwise as apparent from the above discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data and control signals represented as physical (electronic) quantities within the computer system memories or registers or other such information storage, transmission or display devices.

The foregoing invention has been described in accordance with the relevant legal standards, thus the description is exemplary rather than limiting in nature. Variations and modifications to the disclosed embodiment may become apparent to those skilled in the art and fall within the scope of the invention.

What is claimed is:

1. An intra-operative medical image viewing system comprising:
an image source having at least one image file representative of an anatomical feature of a patient;
a display positionable between a surgeon and the patient during surgery, said display being configured to exhibit images to the surgeon overlaid on the patient;
an image control unit configured to retrieve the at least one image file from said image source and control said display to exhibit and modify at least a portion of the at least one image; and
a plurality of peripheral devices each configured to receive an image control input from the surgeon and in response generate an image control signal in a respective user-interface modality, the image control input representative of a desire by the surgeon to modify the at least one image exhibited by said display, wherein each of said plurality of peripheral devices defines a different user-interface modality.

2. The intra-operative medical image viewing system of claim 1 further comprising:
a translator module configured to receive the image control signals from said plurality of peripheral devices in different user-interface modalities, to convert the respective image control signals into an image manipulation command in a common operating room viewer language, and to transmit the image manipulation command to said image control unit, wherein said image control unit controls said display with the image manipulation command received from said translator module.

3. The intra-operative medical image viewing system of claim 2 wherein said image control unit and said translator module are further defined as part of a computing device comprising one or more processors and a non-transitory, computer readable medium storing instructions.

4. The intra-operative medical image viewing system of claim 3 wherein at least one of said one or more processors of said computing device is integral with each of said plurality of peripheral devices and is configured to convert the image control input into the image manipulation command in the common operating room viewer language and transmit the image manipulation command to another of said at least one of said one or more processors of said computing device.

5. The intra-operative medical image viewing system of claim 1 wherein said image control unit is further defined as configured to control said display to modify the at least one image overlaid on the patient by at least one of panning, zooming, rotating, and adjusting the transparency of the overlaid image.

6. The intra-operative medical image viewing system of claim 1 wherein said plurality of peripheral devices includes a microphone configured to receive voice inputs from the surgeon and a motion sensor configured to capture hand-gesture inputs from the surgeon.

7. The intra-operative medical image viewing system of claim 1 wherein said display is further defined as wearable by the surgeon.

8. The intra-operative medical image viewing system of claim 1 wherein said display is further defined as mounted on a frame and sized to be non-wearable by the surgeon.

9. The intra-operative medical image viewing system of claim 1 wherein said display is further defined as a projection directly onto the surface of the patient.

10. An intra-operative medical image viewing system comprising:
an image source having at least one image file representative of an anatomical feature of a patient;
a selectively transparent display positionable between a surgeon and the patient during surgery, said display being configured to exhibit at least one image to the surgeon overlaid on the patient;
an image control unit configured to retrieve the at least one image file from said image source and control said display to exhibit and modify at least a portion of the at least one image, the at least one image being a visual representation of the anatomical feature of the patient;
at least one peripheral device configured to receive an image control input from the surgeon and in response transmit an image control signal to said image control unit, the image control input representative of a desire by the surgeon to modify the at least one image exhibited by said display; and
wherein said image control unit is configured to modify the image in response to the image control signal in any one of a plurality of different three-dimensional modalities.

11. The intra-operative medical image viewing system of claim 10 wherein the image is further defined as two-dimensional and one of said plurality of different three-dimensional modalities is defined as changing an observable plane of the image to add the appearance of depth.

12. The intra-operative medical image viewing system of claim 10 wherein the image is further defined as two-dimensional and one of said plurality of different three-dimensional modalities is defined as wrapping the image around the anatomical feature of the patient.

13. The intra-operative medical image viewing system of claim 10 wherein the image is further defined as a series of three-dimensional tomographic slices of the anatomical feature of the patient and one of said plurality of different three-dimensional modalities is defined as sequentially exhibiting a series of three-dimensional tomographic slices.

14. The intra-operative medical image viewing system of claim 10 wherein the image is further defined as a three-dimensional holographic image generated by tomography of the anatomical feature of the patient.

15. The intra-operative medical image viewing system of claim 10 wherein the image is further defined as three-dimensional, wherein said display is further defined as a binocular viewer, and one of said plurality of different three-dimensional modalities is further defined as a stereoscopic feed present through said binocular viewer.

16. The intra-operative medical image viewing system of claim 10 wherein said at least one peripheral device is further defined as:
a plurality of peripheral devices each configured to receive an image control input from the surgeon and in response generate an image control signal in a respective user-interface modality, the image control input representative of a desire by the surgeon to modify the at least one image exhibited by said display, wherein each of said plurality of peripheral devices defines a different user-interface modality.

17. The intra-operative medical image viewing system of claim 10 wherein the image is further defined as a live stream of intra-operative fluoroscopic imagery.

18. An intra-operative medical image viewing system comprising:
an image source having at least one image file representative of an anatomical feature of a patient;
a selectively transparent display positionable between a surgeon during surgery on the patient, said display being configured to exhibit an image to the surgeon overlaid on the patient;
an image control unit configured to retrieve the image file from said image source and control said display to exhibit and modify the image, the image being a visual representation of the anatomical feature of the patient, wherein said image control unit is responsive to inputs from the surgeon to modify the image to allow the surgeon to selectively position, size and orient the image exhibited on said display to a selectable first configuration; and
a station-keeping module including:
a position module configured to detect a first position of said display when the first configuration is selected and determine a change in position of said display from the first position;
an orientation module configured to detect a first orientation of said display when the first configuration is selected and determine a change in orientation of said display from the first orientation;
a registration module configured to determine a registration default condition defined by the first configuration, the first position, and the first orientation; and
an image recalibration module configured to determine one or more image modification commands to be applied by said display to change the image from the first configuration to a second configuration in response to at least one of the change in position and change in the orientation, said image recalibration module configured to transmit the one or more image modification commands to said image control unit and said image control unit to control said display, the second configuration different from the first configuration and consistent with the registration default condition.

17. The intra-operative medical image viewing system of claim 18 wherein the image includes portions indicating a three-dimensional nature of the anatomical feature of the patient.

21. The intra-operative medical image viewing system of claim 18 wherein said position module is wearable by surgeon.

22. The intra-operative medical image viewing system of claim 18 further comprising:
a plurality of peripheral devices each configured to receive an image control input from the surgeon and in response generate an image control signal in a respective user-interface modality, the image control input representative of a desire by the surgeon to modify the at least one image exhibited by said display, wherein each of said plurality of peripheral devices defines a different user-interface modality.
